(19) United States
(12) Patent Application Publication
(10) Pub. No.: US 2019/0232584 A1
(43) Pub. Date: Aug. 1, 2019

(54) METHOD FOR PRODUCING SUSTAINED DRUG-RELEASE CONTACT LENS

(71) Applicant: SOGANG UNIVERSITY RESEARCH FOUNDATION, Seoul (KR)

(72) Inventors: Jung Wook KIM, Seoul (KR); Hyun Cheol KIM, Seoul (KR)

(21) Appl. No.: 15/761,761

(22) PCT Filed: Sep. 23, 2016
(86) PCT No.: PCT/KR2016/010653

§ 371 Date: Mar. 20, 2018

(30) Foreign Application Priority Data
Sep. 23, 2015 (KR) 10-2015-0134522

(51) Int. Cl. B29D 11/00; A61K 9/00; A61K 47/36; A61K 9/16

(52) U.S. Cl. CPC B29D 11/00096; B29D 11/00134; A61K 9/0051; B29K 2089/00; A61K 9/1658; A61K 9/1617; B29K 2995/006; A61K 47/36

(57) ABSTRACT

The present invention relates to a method of manufacturing a sustained drug-release contact lens. More specifically, the present invention relates to a method of manufacturing a sustained drug-release contact lens, the method including: forming a body in which the body provides an outer shape of the lens and has a plurality of cavities configured to be recessed and spaced apart from each other by a predetermined distance along a side surface of the contact lens; and filling a drug and forming a closing part in which the cavities of the body formed at the forming the body are filled with the drug and respective closing parts closing the cavities are formed, wherein the closing part is made of a biodegradable material and each closing part is configured to open at a different time during wearing of the contact lens at the forming the body.

### CROSS-REFERENCE TO RELATED APPLICATIONS

This application claims the benefit under 35 U.S.C. section 371, of PCT International Application No.: PCT/KR2016/010653, filed on Sep. 23, 2016, which claims foreign priority to Korean Patent Application No.: KR10-2015-0134522, filed on Sep. 23, 2015, in the Korean Intellectual Property Office, both of which are hereby incorporated by reference in their entireties.

### TECHNICAL FIELD

The present invention relates to a method of manufacturing a sustained drug-release contact lens. More specifically, the present invention relates to a method of manufacturing a sustained drug-release contact lens, the method including: forming a body in which the body provides an outer shape of the lens and has a plurality of cavities configured to be recessed and spaced apart from each other by a predetermined distance along a side surface of the body; and filling a drug and forming a closing part in which the cavities of the body formed at the forming the body are filled with the drug and respective closing parts closing the cavities are formed, wherein the closing part is made of a biodegradable material and each closing part is configured to open at a different time during wearing of the contact lens at the forming the body. Thus, the contact lens releases a predetermined amount of the drug continuously, does not release the drug during storage as the closing parts biodegrade due to an enzyme contained in tears, and can be reloaded with the drug.

### BACKGROUND ART

In general, administering eye drop to an eye is widely used to treat eye diseases such as glaucoma. However, it is inconvenient to administer the eye drop regularly, and it is difficult to maintain a concentration of the eye drop in the eye constantly, whereby a treatment effect decreases. Accordingly, to solve such problems, a contact lens capable of releasing drug as in the following Patent Document has been developed.

<Patent Document>

Korean Patent No. 10-1371685, entitled “Therapeutic contact lens”, filed Mar. 3, 2014

However, according to Fick's first law, the amount of diffusion is proportional to a concentration gradient between a drug carrier and an external environment. As a drug release continues in a conventional drug-release contact lens, the concentration gradient decreases. Thus, eye diseases are not treated effectively because the amount of released drug decreases rapidly and the predetermined amount of drug is not possible to be released.

### DISCLOSURE

### Technical Problem

Accordingly, the present invention has been made keeping in mind the above problems occurring in the related art, and

an object of the present invention is to provide a method of manufacturing a sustained drug-release contact lens, which releases a predetermined amount of drug continuously.

In addition, another object of the present invention is to provide a method of manufacturing a sustained drug-release contact lens, which does not release the drug during storage.

Furthermore, still another object of the present invention is to provide a method of manufacturing a sustained drug-release contact lens, which is reloaded with the drug.

### Technical Solution

In order to accomplish the above object, the present invention is implemented according to embodiments having the following constructions.

According to an embodiment of the present invention, a method of manufacturing a sustained drug-release contact lens includes: forming a body in which the body provides a shape of a contact lens and has a plurality of cavities, which is configured to be recessed and spaced a predetermined distance apart from each other along a side surface of the body; and filling a drug and forming a closing part in which the cavities of the body formed at the forming the body are filled with the drug and closing parts which close respective entrances of the cavities are formed.

According to another embodiment of the present invention, the closing part may be made of a biodegradable material and each closing part of the cavities is configured to open at a different time during wearing of the contact lens at the filling the drug and forming the closing part.

According to still another embodiment of the present invention, at the filling the drug and forming the closing part, a solution containing a biodegradable polymer providing the closing part, nanoparticle loaded with the drug, and a photoinitiator may be injected into the cavities through each entrance of the cavities of the body, and the solution may be irradiated with ultraviolet light to photopolymerize such that the contact lens is filled with the drug and provided with the closing part.

According to still another embodiment of the present invention, at the filling the drug and forming the closing part, the solution may be irradiated with ultraviolet light in an asymmetrical annular shape to form different sizes of the cavities whereby each of the cavities has a different degree of closure.

According to still another embodiment of the present invention, at the filling the drug and forming the closing part, each closing part may be configured to have a different decomposition speed by adjusting ultraviolet flux which irradiates the solution for a corresponding cavity.

According to still another embodiment of the present invention, the nanoparticle loaded with the drug may be manufactured by adding a predetermined concentration of the drug to an albumin solution in which albumin is dissolved in distilled water and titrated to a predetermined pH, slowly adding ethanol for desolvation to the solution while stirring, adding a small amount of glutaraldehyde to crosslink particles in the solution after the desolvation process, and stirring the solution at a constant speed.

According to still another embodiment of the present invention, N—AcAc chitosan may be used as the biodegradable polymer.

According to still another embodiment of the present invention, the forming the body may include: forming a middle layer having cavity portions configured to be recessed and spaced apart from each other by a predetermined distance, in which an upper surface, a lower surface, and the side surface of the body communicate with each other through the cavity portions; forming an upper layer which provides the upper surface of the body; forming a lower layer which provides the lower surface of the body; and combining the upper layer and the lower layer with the middle layer interposed therebetween, thus forming the body in which the upper layer is disposed on the middle layer and the lower layer is disposed below the middle layer, wherein, at the combining, an upper surface of the cavity portions is closed by the upper layer and a lower surface thereof is closed by the lower layer such that only a side surface thereof is open whereby the cavities are provided in the body.

According to still another embodiment of the present invention, the method may further include: after a user wears the contact lens manufactured at the filling the drug and forming the closing part for a predetermined time and all of the closing parts biodegrade such that the drug in the cavities is released, reloading a solution in the contact lens by injecting the solution into the cavities of the contact lens, the solution containing a biodegradable polymer, nanoparticle loaded with the drug, and a photoinitiator, and by irradiating the solution with ultraviolet light to photopolymerize such that the contact lens is filled with the drug and provided with the closing parts.

### Advantageous Effects

The present invention can exhibit the following effects according to the above embodiments.

A sustained drug-release contact lens manufactured by a method of the present invention releases a predetermined amount of drug continuously.

In addition, the sustained drug-release contact lens manufactured by a method of the present invention does not release the drug during storage.

Furthermore, the sustained drug-release contact lens manufactured by a method of the present invention is reloaded with the drug.

### DESCRIPTION OF DRAWINGS

FIG. 1 is a perspective view of a contact lens according to an embodiment of the present invention;

FIG. 2 is a partially broken cross-sectional view of a contact lens according to the embodiment of the present invention;

FIG. 3 is a partially cutaway plan view of a second separation tube according to another embodiment;

FIGS. 4 to 7 are reference diagrams showing a method of manufacturing a contact lens according to the embodiment of the present invention; and

FIGS. 8 to 10 are reference diagrams showing a drug release process of a contact lens according to the embodiment of the present invention.

### DESCRIPTION OF REFERENCE NUMERALS IN THE DRAWINGS

1: body
2: nanoparticle
3: closing part
11: cavity
111: first cavity
112: second cavity
12: middle layer
13: upper layer
14: lower layer
121: cavity portion

### BEST MODE

Hereinafter, a method of manufacturing a sustained drug-release contact lens according to the present invention will be described with reference to the accompanying drawings. Unless otherwise defined, all terms including technical and scientific terms used herein have the same meaning as commonly understood by one of ordinary skill in the art to which this invention belongs. When terms used herein discord from the commonly understood meaning, the terms will be interpreted as defined herein. In the following description of the present invention, detailed descriptions of known functions and components incorporated herein will be omitted when it may make the subject matter of the present invention unclear. Unless the context clearly indicates otherwise, it will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

A sustained drug-release contact lens will be described in the present invention before a method of manufacturing the sustained drug-release contact lens according to an embodiment of the present invention is described.

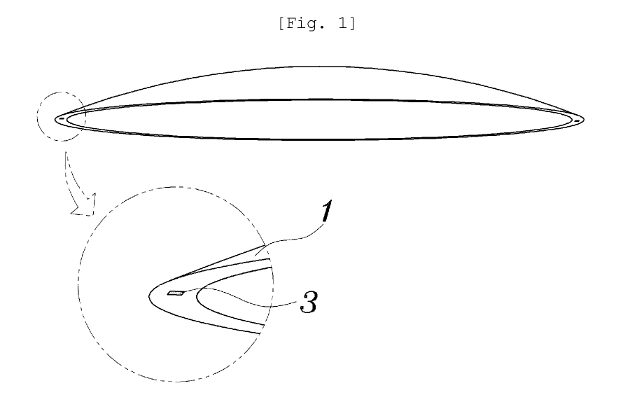

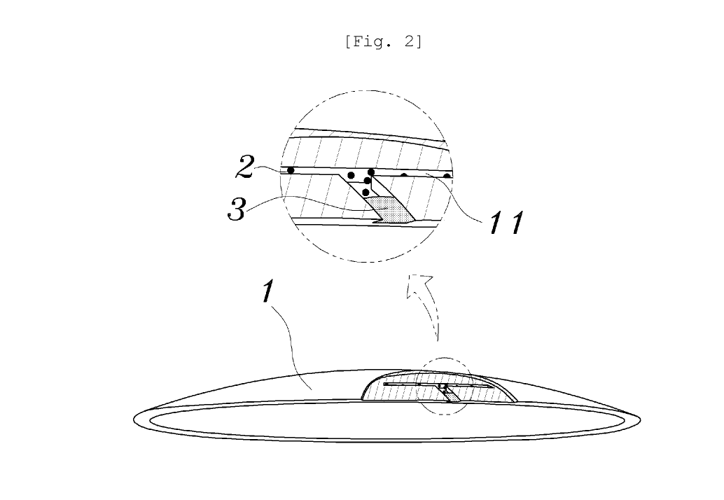

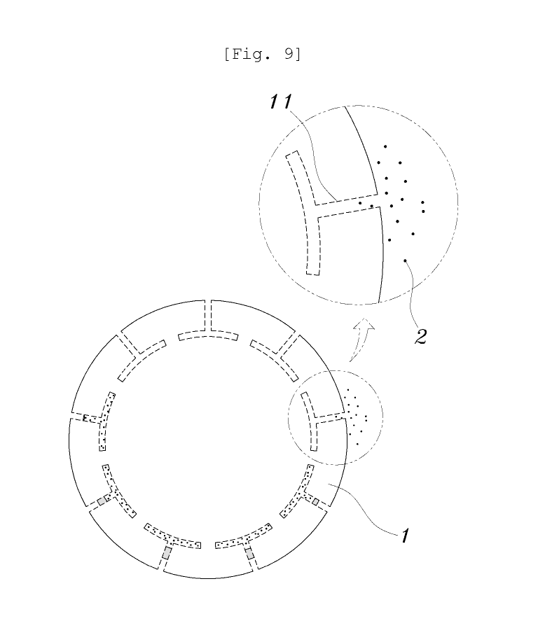

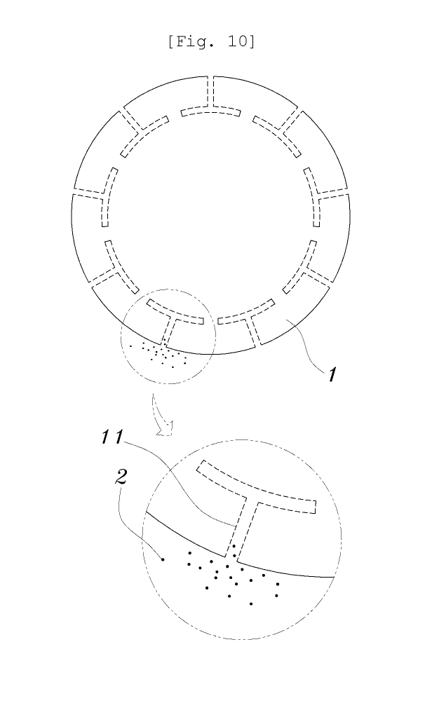

A sustained drug-release contact lens according to an embodiment of the present invention will be described with reference to FIGS. 1 to 10. The contact lens includes a body 1 having cavities 11 configured to be recessed and spaced a predetermined distance apart from each other along a side surface of the body 1; a drug carried in the cavities 11; and a closing part 3 closing each entrance of the cavities 11 where the drug is carried. The closing part 3 biodegrades to open the cavities 11 when the contact lens is worn, and each closing part 3 of the cavities 11 is configured to open at a different time whereby the respective cavities 11 are opened sequentially to release the drug.

The body 1 forms an outer shape of the contact lens, and is provided with a plurality of cavities 11 configured to be spaced a predetermined distance apart from each other along the side surface (outer side surface). The body 1 has an entirely same shape as a contact lens in the related art except that the body 1 has the cavities 11. The body 1 is made of a predetermined material, for example, may be made of a material used for manufacturing a contact lens in the related art, and thus may have micropores of several nanometers as the contact lens in the related art.

The cavities 11 are configured to be recessed in a predetermined depth and spaced a predetermined distance apart from each other along the side surface of the body 1. The body 1 is provided with the plurality of cavities 11, and each cavity 11 carries the drug. The cavities 11 have a predetermined shape, but preferably each cavity 11 includes a first cavity 111 recessed inwardly from the side surface of the body 1, and a second cavity 112 configured to be parallel with the side surface at a predetermined distance and communicate with the first cavity 111 perpendicularly, so the cavities are in a T-shape. In addition, the cavities 11 are disposed to be spaced apart from the center P of the body 1 by a predetermined distance W to prevent the cavities 11, the drug carried on the cavities 11, and the closing parts 3 closing the cavities 11 from being recognized in a visual field of a user during wearing of the contact lens. For example, the distance W greater than a radius of a maximum pupil of the user of the contact lens prevents the cavities 11 from being entered in the visual field of the user.

The drug is carried in the cavities 11. When the contact lens is worn and then each top (entrance) of the cavities 11 is opened by biodegradation of the closing part 3 closing each entrance of the cavities 11, the cavities 11 release the drug into an eyeball of the user. The drug is placed in the cavities 11 as the drug itself, or as a configuration in which the drug is bound or loaded onto various particles. The drug may be in a form in which the drug is bound to particles, for example, a form in which the drug is loaded onto nanoparticle 2 of albumin, which is a representative substance that transports hydrophobic substances in the body among biocompatible substances. The albumin nanoparticle 2 carrying the drug may be 50 nm to 100 nm in diameter. Various substances may be used as the drug for treating eye diseases, for example, latanoprost for treating glaucoma may be used.

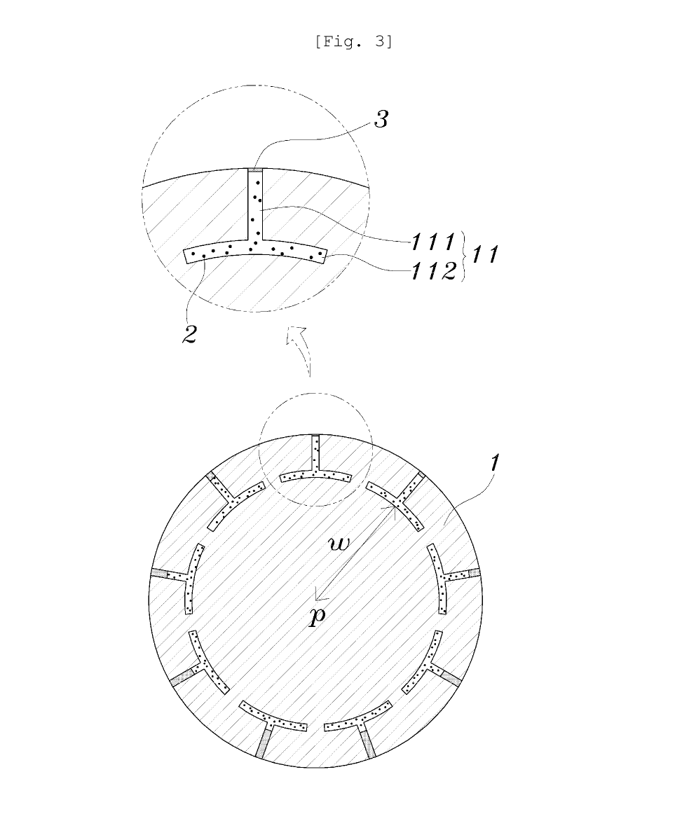

The closing part 3 closes each entrance of the cavities 11 where the drug is carried to prevent release of the drug in the cavities 11 from the cavities 11 during non-wearing of the contact lens. On the other hand, when the user wears the contact lens, the closing part 3 biodegrades to open each entrance of the cavities 11, thereby releasing the drug into the eyeball. The closing part 3 may be composed of various material having biodegradability, as an example, polymer (N—AcAc chitosan), etc. biodegrades due to an enzyme contained in tears (for example, lysozyme, etc.) (N—AcAc chitosan means that polymer made by acetylation (Ac) and acrylation (Ac) of amine group of chitosan in constant proportion). During wearing of the contact lens, the closing part 3 of the respective cavities 11 is configured to open at a different time whereby the respective cavities 11 open sequentially to release the drug. For example, as shown in FIG. 3, each closing part 3 may be configured to have a different size in order to vary a degree of closure (volume) for each of the cavities 11 whereby the respective cavities 11 open at different times. In addition, although it is not described, each closing part 3 is configured to have different decomposition speed whereby the respective cavities 11 open at different times. For example, when a solution contained the polymer (N—AcAc chitosan) is irradiated with ultraviolet light to change information of N-acetylation or N-acrylation of the polymer (N—AcAc chitosan) or to form the closing part 3, the decomposition speed of each closing part 3 varies by adjusting ultraviolet flux for the respective cavities 11.

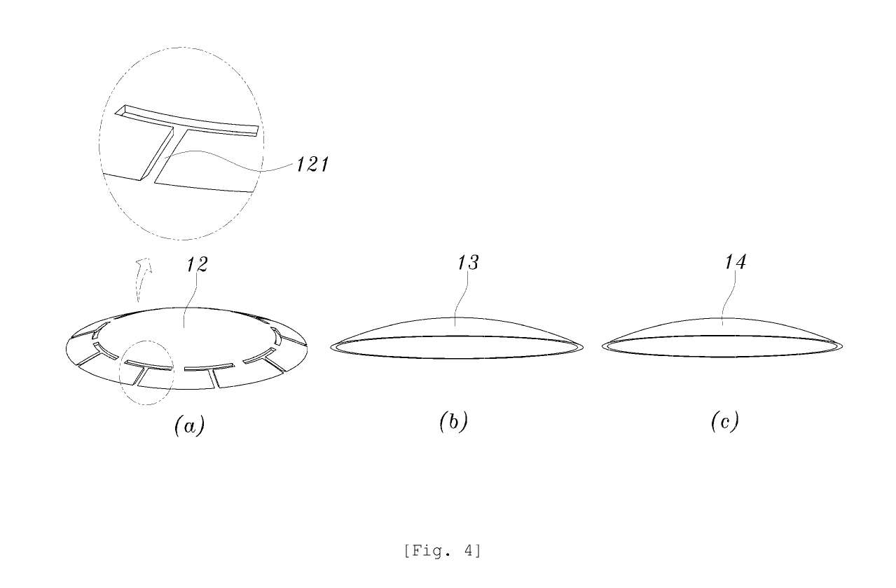

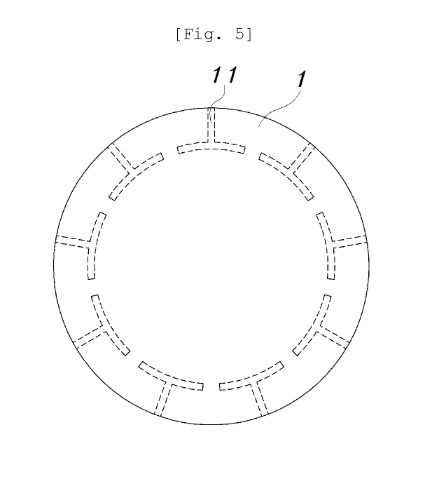

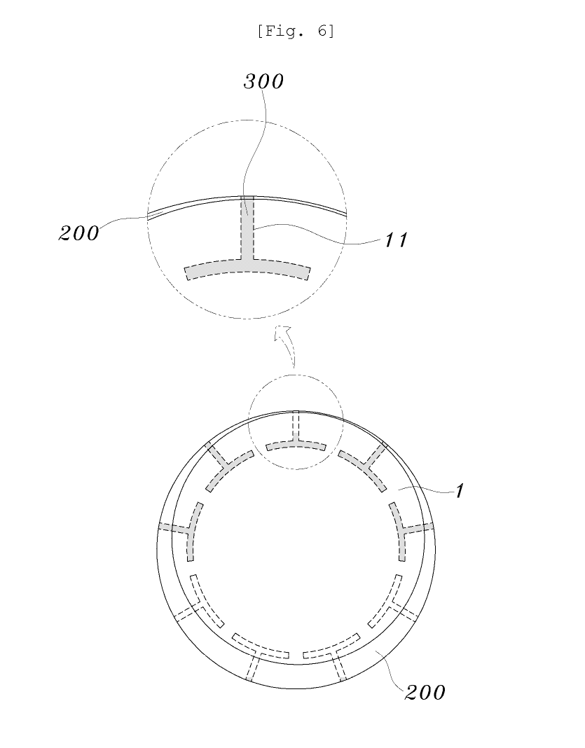

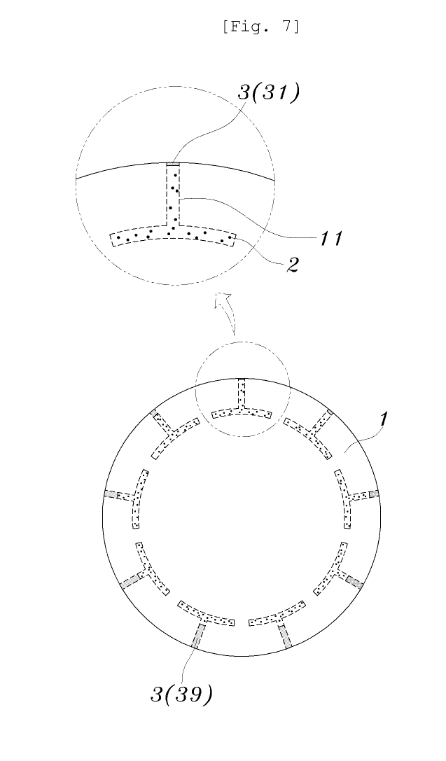

A method of manufacturing the sustained drug-release contact lens having the above-configuration will be described with reference to FIGS. 1 to 7. The method of manufacturing the contact lens includes forming a body, filling the drug and forming the closing part, reloading a solution, and so on. FIG. 4A is a perspective view of a middle layer 12, FIG. 4B is a perspective view of an upper layer 13, and FIG. 4C is a perspective view of a lower layer. FIG. 5 is a plan view of the body 1. FIG. 6 is a plan view showing a process that the cavities 11 of the body 1 is filled with the drug and is irradiated with ultraviolet light to form the closing part 3. FIG. 7 is a plan view showing a manufactured contact lens.

At the forming the body, the body 1 providing a shape of the contact lens and having the plurality of cavities 11 which is configured to be recessed and spaced a predetermined distance apart from each other along the side surface of the body is formed, the forming the body including forming the middle layer, forming the upper layer, forming the lower layer, and combining.

The middle layer 12 is formed at the forming the middle layer, the middle layer having a plurality of cavity portions 121 configured to be recessed and spaced a predetermined distance apart from each other along the side surface. An upper surface, a lower surface, and the side surface of the body communicate with each other through the cavity portions 121 as shown in FIG. 4A. At the forming the middle layer, a solution formed by mixing a monomer used for manufacturing the contact lens (for example, HEMA (2-hydroxyethyl methacrylate), etc.) and cross-linking agent (for example, EGDMA (ethylenegylcol), etc.) is used, and the middle layer 12 is provided with the cavity portions 121 by printing/stamping used for manufacturing the contact lens or photolithography.

The upper layer 13 providing the upper surface (outer surface) of the body 1 is formed at the forming the upper layer, and is manufactured in a same manner as a method manufacturing the contact lens in the related art.

The lower layer 14 providing the lower surface (inner surface) of the body 1 is formed at the forming the lower layer, and is manufactured in a same manner as a method manufacturing the contact lens in the related art.

At the combining, the body 1 is formed by combining the upper layer 13 and the lower layer 14 with the middle layer 12 interposed therebetween as shown in FIG. 5, in which the upper layer 13 is disposed on the middle layer 12 and the lower layer 14 is disposed below the middle layer 12. The upper surface of the cavity portions 121 is closed by the upper layer 13 and the lower surface thereof is closed by the lower layer 14 such that only a side surface thereof is open whereby the cavities 11 are provided in the body 1. The middle layer 12, the upper layer 13, and the lower layer 14 may be combined by various methods, for example, by applying adhesives.

The cavities 11 of the body 1 formed at the forming the body are filled with the drug and the respective closing parts 3 which close each entrance of the cavities 11 are formed at the filling the drug and the forming the closing part. At the filling the drug and forming the closing part, the closing part 3 is composed of a biodegradable material and each closing part 3 of the cavities 11 is configured to open at a different time during wearing of the contact lens.

In specific, a solution 300 containing a biodegradable polymer (N—AcAc chitosan) providing the closing part 3, the drug (or the nanoparticle 2 having 50 nm to 100 nm of diameter and loaded with the drug), and a photoinitiator (PI) is introduced into the cavities 11 through each entrance of the cavities 11 of the body 1, and the solution 300 is irradiated with ultraviolet light (UV) to photopolymerize such that the contact lens filled with the drug and provided with the closing parts 3 is manufactured. At this point, a biodegradable polymer non-participative in the photopolymerization escapes through nanopores of the body 1, then only the nanoparticle 2 having 50 nm to 100 nm of diameter and loaded with the drug remains in the cavities 11. In the above process, when the solution is irradiated with UV in an asymmetrical annular shape 200 such that a thickness of a ring changes depending on a position in the ring (that is, each of cavities 11 is irradiated with a different area or amount of UV) by using a photomask, digital mirror device (DMD), and so on, each size of the closing parts 3 is formed differently whereby each of the respective cavities 11 has a different degree (volume) of closure.

In addition, it is also possible to individually adjust a degree of crosslinking instead of irradiating the solution with UV in the asymmetrical annular shape 200 so that the cavities 11 are opened at different times. Since the closing parts 3 are formed by photopolymerization, each degree of crosslinking of the closing parts 3 is determined according to ultraviolet light flux, and the ultraviolet light flux is controlled by digital light processing (DLP) technique to vary a decomposition speed of the respective closing parts 3.

The nanoparticle 2 loaded with the drug is manufactured by various methods. For example, albumin is dissolved in distilled water, and titrated to a predetermined pH. Then, ethanol for desolvation is slowly added to above dissolved albumin while stirring at room temperature. After the desolvation process, a small amount of glutaraldehyde is added to crosslink the particles in above solution, and the nanoparticle 2 is completed with stirring at a constant speed. The loading of the drug is carried out in such process in which a predetermined concentration of drug solution is added to the albumin solution which is a first solution, the mixture is stirred for 24 hours to bind to hydrophobic part of the albumin, and when the albumin is aggregated by the addition of the ethanol for the desolvation to become the nanoparticle, the drug is loaded between matrixes of proteins by aggregation. The drug which is not loaded onto the nanoparticle 2 and non-nanoparticle albumin are isolated by centrifugation, and thus the albumin nanoparticle 2 loaded with the drug and having 50 nm to 100 nm of diameter can be manufactured after repeated centrifugation to remove impurities.

After a user wears the contact lens manufactured at the filling the drug and the forming the closing part for a predetermined time and all of the closing parts 3 biodegrade such that the drug in the cavities 11 is released, at the reloading a solution, the cavities of the contact lens in which the closing parts 3 biodegraded and all of the drug was released are injected with the solution 300 containing the biodegradable polymer (N—AcAc chitosan), the drug (or the nanoparticle 2 having 50 nm to 100 nm of diameter and loaded with the drug), and a photoinitiator (PI), and the solution 300 is irradiated with ultraviolet light (UV) to photopolymerize such that the contact lens filled with the drug and provided with the closing parts 3 is manufactured as same with the contact lens manufactured at the filling the drug and forming the closing part. Because the reloading is performed in a same manner with the filling the drug and forming the closing part except using a used contact lens (body 1), a detail description will be omitted.

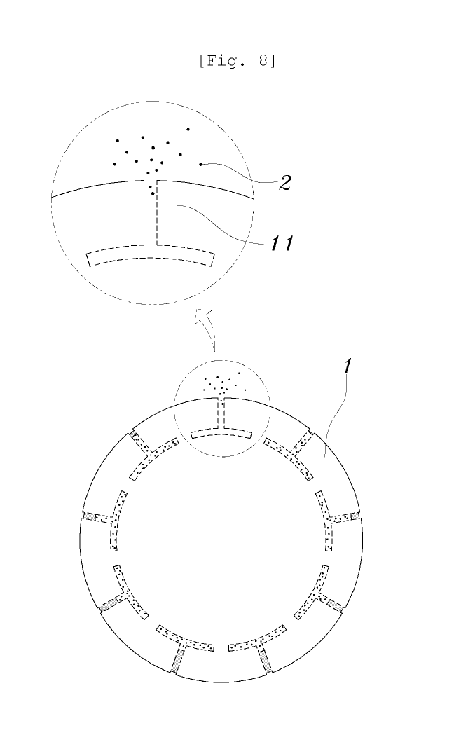

A drug release process of the sustained drug-release contact lens having the above-described construction and manufactured by the above method will be described with reference to FIGS. 8 to 10. FIGS. 8 to 10 are plan views showing the drug release process in which each of the cavities 11 is opened sequentially in time whereby the drug is released.

When the user wears the contact lens, the biodegradable polymer (N—AcAc chitosan) consisting the closing parts 3 gradually biodegrades due to the enzyme contained in tears (for example, lysozyme, etc.). Since the respective closing parts 3 have different sizes (that is, each of the cavities 11 has a different degree (volume) of closure), a smallest size closing part 31 biodegrades completely first and a largest size closing part 39 biodegrades completely last, that is, the respective cavities 11 are opened sequentially to release a constant amount of the drug continually.

Although the preferred embodiments of the present invention have been disclosed for illustrative purposes, it is well known to those skilled in that art that the present invention is not limited to the embodiment disclosed in the detailed description, and the patent right of the present invention should be defined by the scope and spirit of the invention as disclosed in the accompanying claims. Accordingly, it should be understood that the present invention includes various modifications, additions and substitutions without departing from the scope and spirit of the invention as disclosed in the accompanying claims.

1. A method of manufacturing a sustained drug-release contact lens, the method comprising:
forming a body in which the body provides a shape of a contact lens and has a plurality of cavities, which is configured to be recessed and spaced a predetermined distance apart from each other along a side surface of the body; and
filling a drug and forming a closing part in which the cavities of the body formed at the forming the body are filled with the drug and closing parts which close respective entrances of the cavities are formed.

2. The method of claim 1, wherein the closing part is made of a biodegradable material and each closing part of the cavities is configured to open at a different time during wearing of the contact lens at the filling the drug and forming the closing part.

3. The method of claim 2, wherein, at the filling the drug and forming the closing part, a solution containing a biodegradable polymer providing the closing part, nanoparticle loaded with the drug, and a photoinitiator is injected into the cavities through each entrance of the cavities of the body, and the solution is irradiated with ultraviolet light to photopolymerize such that the contact lens is filled with the drug and provided with the closing part.

4. The method of claim 3, wherein, at the filling the drug and forming the closing part, the solution is irradiated with ultraviolet light in an asymmetrical annular shape to form different sizes of the cavities whereby each of the cavities has a different degree of closure.

5. The method of claim 2, wherein, at the filling the drug and forming the closing part, each closing part is configured to have a different decomposition speed by adjusting ultraviolet flux which irradiates the solution for a corresponding cavity.

6. The method of claim 4, wherein the nanoparticle loaded with the drug is manufactured by adding a predetermined concentration of the drug to an albumin solution in which albumin is dissolved in distilled water and titrated to a predetermined pH, slowly adding ethanol for desolvation to the solution while stirring, adding a small amount of glutaraldehyde to crosslink particles in the solution after the desolvation process, and stirring the solution at a constant speed.

7. The method of claim 6, wherein N—AcAc chitosan is used as the biodegradable polymer.

8. The method of claim 2, wherein the forming the body includes:
forming a middle layer having cavity portions configured to be recessed and spaced apart from each other by a predetermined distance, in which an upper surface, a lower surface, and the side surface of the body communicate with each other through the cavity portions;
forming an upper layer which provides the upper surface of the body;
forming a lower layer which provides the lower surface of the body; and
combining the upper layer and the lower layer with the middle layer interposed therebetween, thus forming the body in which the upper layer is disposed on the middle layer and the lower layer is disposed below the middle layer,
wherein, at the combining, an upper surface of the cavity portions is closed by the upper layer and a lower surface thereof is closed by the lower layer such that only a side surface thereof is open whereby the cavities are provided in the body.

9. The method of claim 3, further comprising:
after a user wears the contact lens manufactured at the filling the drug and forming the closing part for a predetermined time and all of the closing parts biodegrade such that the drug in the cavities is released, reloading a solution in the contact lens by injecting the solution into the cavities of the contact lens, the solution containing a biodegradable polymer, nanoparticle loaded with the drug, and a photoinitiator, and by irradiating the solution with ultraviolet light to photopolymerize such that the contact lens is filled with the drug and provided with the closing parts.
